(19) United States
(12) Patent Application Publication
(10) Pub. No.: US 2009/0297627 A1
(43) Pub. Date: Dec. 3, 2009

(54) Method and composition to improve performance during long term sporting event

(76) Inventor: Darrell L. Tanelian, Dallas, TX (US)

Correspondence Address: TOD R NISSLE, PO BOX 55630, PHOENIX, AZ, 85078, US

(21) Appl. No.: 12/154,765

(22) Filed: May 27, 2008

(51) Int. Cl. A61K 33/30

(52) U.S. Cl. 424/643

(57) ABSTRACT

A process to improve performance of an individual during a long term sporting event includes the steps of providing in ingestible form a plurality of doses of a first nutritional composition, each dose including a known equivalent concentration of calcium, magnesium, potassium, and zinc; providing in ingestible form a plurality of doses of a second nutritional composition, each dose of the second nutritional composition including a known equivalent concentration of calcium, magnesium, potassium, sodium, and zinc; establishing a dietary meal plan including the first nutritional composition for the individual to establish a urine pH in the range of 6.5 to 7.5; following the dietary meal plan to establish a urine pH in the range of 6.5 to 7.5; and, periodically measuring urine pH to confirm a urine pH in the range of 6.5 to 7.5. The process also includes the steps of, during training of the individual prior to the sporting event, generating data during one or more training intervals indicating changes in urine pH during the training interval; administering a selected amount of one or more of the doses of the second nutritional composition after a subsequent training interval, the subsequent training interval extending over a selected period of time; generating data indicating changes in urine pH after the second nutritional composition is administered during the subsequent training interval; and, repeating steps (i) to (iii) to determine a fixed quantity of the doses to maintain urine pH at a selected level in the range of pH 6.5 to pH 7.5 during the sporting event. The process also includes the steps of beginning to compete in the long term sporting event; and, at selected intervals during the long term sporting event, ingesting the fixed quantity of the doses to maintain the urine pH at a selected level in the range of pH 6.5 to pH 7.5.

This invention pertains to long term sporting events.

More particularly, the invention pertains to a training and nutritional regimen prior to and during sporting events.

A principal object of the instant invention is to provide a training and nutritional regimen that will enhance performance during a long term sporting event.

I have discovered a training and nutritional regiment that can enhance performance during a long term sporting event.

As used herein, a long term sporting event is a sporting event that lasts for one hour or more including, for example, marathons and triathlons.

Briefly, in accordance with the invention, I have discovered an improved process for improving an individual=s performance during a long term sporting event. The process comprises the steps of providing in ingestible form a plurality of doses of a first nutritional composition, each dose including a known equivalent concentration of calcium, magnesium, potassium, and zinc; providing in ingestible form a plurality of doses of a second nutritional composition, each dose of the second nutritional composition including a known equivalent concentration of calcium, magnesium, potassium, sodium, and zinc; establishing a dietary meal plan for the individual to establish a urine pH in the range of 6.0 to 8.0, preferably 6.5 to 7.5; following the dietary meal plan to establish a urine pH in the range of 6.0 to 8.0, preferably 6.5 to 7.5; periodically measuring urine pH to confirm a urine pH in the range of 6.0 to 8.0, preferably 6.5 to 7.5; and, generating dose data. Dose data is generated during training of the individual prior to the sporting event, (i) generating data during one or more training intervals indicating changes in urine pH during the training interval; (ii) administering a selected amount of one or more of the doses of the second nutritional composition after completion of a subsequent training interval, said subsequent training interval extending over a selected period of time; (iii) generating data indicating changes in urine pH after administration of the second mineral composition; and, repeating the foregoing steps (i) to (iii) to determine a fixed quantity of the doses to maintain urine pH at a selected level in the range of pH 6.0 to 8.0, preferably pH 6.5 to pH 7.5 during the sporting event. The process also includes the steps of beginning to compete in the long term sporting event; and, at selected intervals during the long term sporting event, ingesting the fixed quantity of the doses to maintain the urine pH at a selected level in the range of pH 6.0 to 8.0, preferably pH 6.5 to pH 7.5.

With respect to the first nutritional composition, calcium, magnesium and zinc are preferably, but not necessarily, in the form of a lactate, while potassium is in the form of a bicarbonate. Each dose of the first nutritional composition is presently preferably in powder form and, by way of example and not limitation, includes 1120 mg of calcium, 350 mg of magnesium, seven milligrams of zinc and 1170 mg of potassium. The powder is, at the time of administration, admixed with water, juice, etc. to produce a liquid that is ingested by an individual. Alternatively, the powder can be admixed with water, juice, etc. to produce a liquid prior to the time the powder is to be ingested by an individual. The powder can be stored in a container in bulk, in capsules, or in any other desired configuration. As would be appreciated by those of skill in the art, the minerals can be administered in forms other than a liquid, including by way of example and not limitation, in a gel, in a candy bar, in a nutritional bar, and as a powder admixed with sugar granules. In one dose of the first nutritional composition, the amount of calcium can comprise from twenty to 2000 mg; the amount of magnesium can comprise from fifty to 500 mg; the amount of zinc can comprise from one to fifty mg; and, the amount of potassium can comprise from one to 4800 mg.

With respect to the second nutritional composition, calcium, magnesium and zinc are preferably, but not necessarily, in the form of a lactate, while potassium and sodium are in the form of a bicarbonate. Each dose of the second nutritional composition is presently preferably in powder form and, by way of example and not limitation, includes 43 mg of calcium, 10 mg of magnesium, one milligram of zinc, 300 mg of potassium, and 500 mg of sodium. The powder is, at the time of administration, admixed with water, juice, etc. to produce a liquid that is ingested by an individual. Alternatively, the powder can be admixed with water, juice, etc. to produce a liquid prior to the time the powder is to be ingested by an individual. The powder can be stored in bulk in a container, in capsules, or in any other desired configuration. The quantities of calcium, magnesium, zinc, and potassium in the second nutritional composition are much less than in the first nutritional composition because the second nutritional composition is administered during a sporting event after significantly shorter periods of time, for example an hour, have elapsed during the sporting event. Further, in contrast to the first nutritional composition, the second nutritional composition includes sodium. As would be appreciated by those of skill in the art, the minerals can be administered in forms other than a liquid, including by way of example and not limitation, in a gel, in a candy bar, in a nutritional bar, and as a powder admixed with sugar granules. In one dose of the second nutritional composition, the amount of calcium can comprise from twenty to 100 mg; the amount of magnesium can comprise from five to thirty mg; the amount of zinc can comprise from one-tenth to five mg; the amount of potassium can comprise from one to 480 mg; and, the amount of sodium can comprise from one to 1500 mg.

With respect to establishing a dietary meal plan for the individual to establish a urine pH in the range of 6.5 to 7.5, such dietary plans are available. In general, most fruits and vegetable and drinks produce an alkaline urine pH, while most proteins, starches (grains), and milk products produce an acidic urine pH. For example, raisins and spinach produce high alkaline pH readings, while cheese and meat products produce high acidic pH readings. A dietary meal plan can readily include cheese, starch, and meat as long as such foods are offset by an intake of alkaline foods. For example:

TABLE I

| BREAKFAST A | | BREAKFAST B | |
|---|---|---|---|
| Orange Juice, | −6.63* | Milk, 8 oz | 1.6 |
| 8 oz | | | |
| Coffee, 8 oz | −3.2 | Coffee, 8 oz | −3.2 |
| Tomato, 3.5 oz | −3.1 | Wheat Toast, 2 slices | 1.8 |
| Potato, 3.5 oz | −4.0 | Corn Beef, 3.5 oz | 13.2 |
| 2 Eggs | 8.2 | 2 Eggs | 8.2 |
| TOTAL | −8.73 | TOTAL | 21.6 |

*Negative values (i.e., orange juice) indicate a food that produces an alkaline urine pH. Positive values (i.e., milk) indicate a food that produces an acidic urine pH.

TABLE II

| DINNER A | | DINNER B | |
|---|---|---|---|
| Mineral Water, 12 oz. | −6.5* | Tap Water | 0 |
| Red Wine, 7.3 oz | −4.8 | Beer, 12 oz | −0.34 |
| Potato, 7 oz | −8.0 | Pasta 7 oz | 14.6 |
| Steak (Lean), 7 oz | 15.6 | Steak (Lean), 7 oz | 15.6 |
| Spinach, 3.5 oz | −14.0 | Broccoli, 3.5 oz | −1.2 |
| TOTAL | −17.7 | TOTAL | 28.66 |

*Negative values (i.e., orange juice) indicate a food that produces an alkaline urine pH. Positive values (i.e., milk) indicate a food that produces an acidic urine pH.

Accordingly, assuming that an individual prior to eating breakfast has a urine pH of 7.0 or more, eating Breakfast A in Table I and Dinner A in Table II will facilitate achieving an alkaline urine pH reading of 7.0+, while eating Breakfast B in Table I and Dinner B in Table II will facilitate achieving acidic urine pH readings of less than 7.0. While it is possible that the particular pH effect may vary to a certain extent from individual to individual, one virtue of utilizing urine pH is that it is readily and conveniently tested using pH strips. A pH strip with one or two indicators can be utilized, but pH strips with three or four indicators per strip are preferred because they are more accurate. The pH strips preferably are utilized three to four hours after a meal because an individual's body will have had sufficient time to digest the meal.

Once an individual has, in the manner noted above, established a diet that consistently produces a urine pH in the range of 6.0 to 8.0 preferably 6.5 to 7.5, most preferably 7.0 to 7.5, then additional data is generated during training. First, data indicating changes in urine pH are developed by testing the urine pH before and after a selected training interval. The training interval selected can vary as desired, but one hour is selected by way of example, and the sporting event selected is the triathlon and the training consists of swimming for one hour. The temperature of the water and speed at which the individual swims are comparable to that encountered during the actual triathlon. The individual's urine pH prior to the training interval is pH 7.4. After swimming for one hour, the individual's urine pH is 6.8. One dose, or packet, of the second nutritional composition includes 43 mg of calcium, 10 mg of magnesium, one milligram of zinc, 300 mg of potassium, and 500 mg of sodium. One packet of the second nutritional composition is mixed with water and ingested by the individual. After three hours, the individual's urine pH is 7.2. The urine is tested after three hours in order to give the body time to metabolize the minerals. As a result, it is decided to administer one and one-half doses, i.e., one and one-half packets after the next training session. Prior to the next training session, the individual's urine pH is 7.3. The next training session is a one hour swim. The temperature of the water and speed at which the individual swims are comparable to that encountered during the actual triathlon. After the training session, the individual's urine pH is 6.8 and one and one-half packets are mixed with water, orange juice or another desired liquid and ingested. After three hours, the individual's urine pH is 7.4. Based on this data, it is decided to administer one and one-half packets to the individual after each hour of swimming. During the swimming portion of the triathlon, one and one-half packets of the second nutritional composition are mixed in orange juice (or water, apple juice, etc.) and ingested by the individual after each hour of swimming.

### EXAMPLE 1

In September 2007, sixteen weeks prior to the Chevron Houston Marathon on Jan. 8, 2008, fifteen subjects are recruited. The urine pH of each subject is measured with a three indicator pH strip. Age and prior marathon times are determined. This data is set forth below in Table III.

TABLE III

| | | | MARATHON TIME, |
|---|---|---|---|
| | | | CHEVRON HOUSTON |
| | | URINE | MARATHON January 2007* |
| SUBJECT | AGE | PH AT OUTSET | (Hours:Minutes:Seconds) |
| 1 | 22 | 5.2-5.6 | 3:40:22 |
| 2 | 24 | 5.3-5.7 | 3:56:46 |
| 3 | 25 | 5.4-5.8 | 4:08:12 |
| 4 | 26 | 5.3-5.7 | 3:31:19 |
| 5 | 27 | 5.6-6.0 | 2:48:46 |
| 6 | 28 | 5.2-5.6 | 2:51:33 |
| 7 | 32 | 5.4-5.8 | 3:30:42 |
| 8 | 36 | 5.2-5.6 | 3:52:06 |
| 9 | 39 | 5.3-5.7 | 3:43:53 |
| 10 | 43 | 5.3-5.7 | 4:20:21 |
| 11 | 44 | 5.1-5.5 | 4:06:34 |
| 12 | 48 | 5.4-5.8 | 3:58:42 |
| 13 | 53 | 5.3-5.7 | 4:32:37 |
| 14 | 56 | 5.2-5.6 | 4:46:20 |
| 15 | 56 | 5.2-5.6 | 5:10:59 |

*Sunny during marathon, high temperature 64 degrees F. Subjects were in good health during the marathon. Time taken by each subject to complete the marathon were comparable to other marathons run by subjects.

The even numbered subjects (2, 4, 6, 8, 10, 12, 14) comprise the control group and prepare for the next marathon (to be run in January 2008) in their normal manner.
The odd numbered subjects (1, 3, 5, 7, 9, 11, 13, 15) comprise the test group and prepare for the next marathon (to be run in January 2008) in the following manner:
1. Each subject in the test group ingests a nutritional diet to produce daily a urine pH in the range of 6.5 to 7.5. The urine pH is tested with three indicator urine pH strips once daily three to four hours after the dinner (late afternoon) meal. As part of their daily nutritional diet, each individual in the test group consumes daily one to two packets of powder of the first nutritional composition. Each packet includes 1120 mg of calcium, 350 mg of magnesium, seven milligrams of zinc and 1170 mg of potassium. The calcium, zinc, and magnesium are lactates. The potassium is a bicarbonate. The powder is mixed with eight to twelve ounces of water (or any desired beverage) prior to being consumed.
2. Each subject maintains his own 16 week training schedule, but agrees on Thursday and Sunday of each week to include the training as set forth below in Table IV. The training set forth in Table I is, along with recommended training on the other days of the week, a commonly recommended training schedule on Thursday and Sunday for intermediate marathon runners, and is comparable to the training each subject undertook prior to the January 2007 marathon of Table IV.

TABLE IV
Thursday, Sunday Training Schedule

| | | Sunday | |
|---|---|---|---|
| Week | Thursday Training | Training | |
| 1* | 1 hour run, including four to five minutes total | 8 | miles |
| | uphill time (TUT) | | |
| 2* | 1 hour run, including four to five minutes total | 10 | miles |
| | uphill time (TUT) | | |
| 3* | 70 minute run, including five to six minutes total | 12 | miles |
| | uphill time (TUT) | | |
| 4* | 70 minute run, including five to six minutes total | 14 | miles |
| | uphill time (TUT) | | |
| 5 | 4 × 800 | 10 | miles |
| 6* | 80 minute run, including six to eight minutes total | 15 | miles |
| | uphill time (TUT) | | |
| 7* | 80 minute run, including six to eight minutes total | 16 | miles |
| | uphill time (TUT) | | |
| 8 | 4 × 1 mile | 16 | miles |
| 9 | 4 × 1 mile | 17 | miles |
| 10 | 4 × 800; 6 × 100 | 6 to 8 | miles |
| 11* | 90 minute run, including eight to ten minutes total | 18 | miles |
| | uphill time (TUT) | | |
| 12* | 90 minute run, including eight to ten minutes total | 19 | miles |
| | uphill time (TUT) | | |
| 13* | 75 minute run, including six to eight minutes total | 20 | miles |
| | uphill time (TUT) | | |
| 14* | 75 minute run, including six to eight minutes total | 13 | miles |
| | uphill time (TUT) | | |
| 15 | One hour run, including 6 × 400 | 1 | hour |

| | | |
|---|---|---|
| 16 | Three miles; 6 × 100 | Marathon |

*During these weeks, on Thursday urine pH is tested just prior to the run (i.e., the pre-run urine pH), at the end of the run, and three to four hours after end of the run. To compensate for the reduction in urine pH that occurs during the run from one-half to two of the second nutritional composition powder packets (where each packet comprises a “dose”) are admixed with orange juice and ingested each hour during the run and at the end of the run and the urine pH (i.e., the recovery urine pH) is tested three to four hours after the end of the run to determine if the urine pH has returned to the pre-run urine pH (i.e., if the recovery urine pH equals the pre-run urine pH). If, of course, the run lasts less than one hour, then the second nutritional composition is administered only at the end of the run. If the period of time that elapses prior to a dose is less than an hour, then the size of the dose is adjusted proportionately. For example, if it is determined to administer one dose after an hour of running and the duration of the run is one and one-half hours, then after the first hour of the run one dose is administered, and after the last one-half hour of the run only one-half of a dose is administered. The data produced during the foregoing procedure is stored each week and the quantity of the second nutritional powder is adjusted until the quantity of second nutritional powder appears sufficient for the recovery urine pH to equal the pre-run urine pH. For example, if in week 1 the pre-run urine pH is 7.2, the post-run urine pH is 6.8, one-half packet of the second nutritional powder is ingested, and three hours later the recovery urine pH is 7.0, then it appears a sufficient quantity of the second nutritional powder was not administered. Consequently, if in week 1 the pre-run urine pH is 7.2 and the post-run urine pH is 6.8, then a full packet of the second nutritional powder is ingested. If the recovery pH is then 7.3, then it appears that only ¾ of a packet of the second nutritional powder is sufficient. If these data are maintained during the sixteen week training program, a good estimate is obtained for each member of the test group as to the amount of the second nutritional powder that should be ingested after each hour of a marathon.

At the completion of the sixteen week training, each of the fifteen subjects competes in the Jan. 13, 2008 Chevron Houston Marathon. The urine pH of each subject is tested just prior to running the marathon (pre-run urine pH). Each member of the test group is, after each hour of running the marathon, given a quantity of the second nutritional powder as determined above. At the end of the marathon, the urine pH (post run urine pH) of each of the fifteen subjects is tested. This data is set forth in Table V below.

TABLE V

| | | | | MARATHON |
|---|---|---|---|---|
| | | | | TIME, |
| | | | | CHEVRON |
| | | POST- | MARATHON TIME, | HOUSTON |
| | | RUN | Chevron Houston | MARATHON |
| | PRE-RUN | URINE | Marathon January 2008* | January 2007 |
| Subject | URINE pH | pH | (Hours:Minutes:Seconds) | (Hours:Minutes:Seconds) |
| 1 | 7.0-7.4 | 6.9-7.3 | 3:37:46 | 3:40:22 |
| 2 | 5.3-5.7 | 4.7-5.1 | 3:55:03 | 3:56:46 |
| 3 | 7.0-7.4 | 7.0-7.4 | 4:04:23 | 4:08:12 |
| 4 | 5.2-5.6 | 5.7-5.1 | 3:33:36 | 3:31:19 |
| 5 | 7.2-7.6 | 6.9-7.3 | 2:46:48 | 2:48:46 |
| 6 | 5.3-5.7 | 4.8-5.2 | 2:52:40 | 2:51:33 |
| 7 | 6.8-7.2 | 6.6-7.0 | 3:24:21 | 3:30:42 |
| 8 | 5.2-5.6 | 4.6-5.0 | 3:51:04 | 3:52:06 |
| 9 | 6.7-7.1 | 6.6-7.0 | 3:35:17 | 3:43:53 |
| 10 | 5.4-5.8 | 4.7-5.1 | 4:22:38 | 4:20:21 |
| 11 | 7.3-7.7 | 6.9-7.3 | 3:59:46 | 4:06:34 |
| 12 | 5.4-5.8 | 4.8-5.2 | 3:57:32 | 3:58:42 |
| 13 | 7.2-7.6 | 7.0-7.4 | 4:26:28 | 4:32:37 |
| 14 | 5.4-5.8 | 4.7-5.1 | 4:48:26 | 4:46:20 |
| 15 | 7.0-7.4 | 6.6-7.0 | 4:58:55 | 5:10:59 |

*Sunny during marathon, high temperature 66 degrees F. Subjects were in good health during the marathon.

The use of the second nutritional composition in combination with an alkaline urine pH are initially being investigated as a way to replace electrolytes during a sporting event. The significant improvement in performance by the subjects in the test group is unexpected and unpredicted.

### EXAMPLE II

Example I is repeated, except that the subjects are training for the running leg of a triathlon. Similar significant improvements in performances of the test group are achieved.

### EXAMPLE III

Example I is repeated, except that the subjects are training for a long term bicycle race. Similar significant improvements in performances of the test group are achieved.

Judicial Notice is Taken of the Following Facts:
1. The majority of Americans have an acidic urine pH of less than 6.0, typically in the range of 4.5 to 5.9, probably 5.0 to 5.6. One well known cause of such a pH is the lack of minerals in the processed foods that are eaten in the United States. Another well known cause of such a pH is that crops are grown in mineral depleted soil and, as a result, the food produced by the crops is lacking in mineral content. Another important cause of such a pH is that most people ingest an acid generating diet.
2. There existed at the time of the invention a dominant, long felt trend that did not address or recognize urine pH as being important to an athlete's performance in a long term sporting event. Millions of athletes have for many years been trained for a variety of sporting events without any focus on urine pH during and at the start of athletic events.
3. A countervailing subservient trend with respect to said dominant trend in 2. above did not appear to exist at the time of the invention.
4. There existed at the time of the invention a dominant, long felt trend that did not address or recognize mineral supplements as being important to an athlete's performance in a long term sporting event.
5. A countervailing subservient trend with respect to said dominant trend in 4. above did not appear to exist at the time of the invention.
6. There existed at the time of the invention a dominant, long felt trend that did not address or recognize the use of minerals during a sporting event to control the urine pH of an athlete.
7. A countervailing subservient trend with respect to said dominant trend if 8. above did not did not appear to exist at the time of the invention.
8. There did not exist at the time of the invention a recognized problem, market need, or motivation that provided significant impetus to develop the invention.
9. There did not exist at the time of the invention a recognized problem to which there was a set of specific solutions, one of which was the invention.
10. There did not exist at the time of the invention a process in which a high urine pH in the range of 6.5 to 7.5 was produced prior to a long term athletic event and was maintained during the athletic event.
11. Common sense is judgment that requires valid reasoning supporting and justifying such judgment.
12. One of ordinary skill in the art with respect to the invention herein has specialized knowledge over and above the baseline knowledge of the general population, which specialized knowledge is in connection with blood chemistry, nutrition, and sports training.
13. The TSM test can provide helpful insight into whether an invention is obvious.
14. The broad general motivation to make a product or process better is common to every invention.
15. There normally are broad commonplace motivations with respect to each particular class of invention. For example, one commonplace motivation with respect to exercise equipment is to make it versatile.
16. The existence of a broad general motivation, without more, does not necessarily provide any significant impetus to produce an invention.
17. A specific problem, motivation, or market trend is more likely to produce significant impetus to produce an invention than a commonplace motivation. If, for example, a piece of exercise equipment causes a greater than normal quantity of injuries, that is more likely to produce significant impetus to produce an invention that the commonplace motivation of making equipment better.
18. A problem may not provide significant impetus for an invention if the problem suggests solutions other than the invention.
19. At the time of the invention, blood serum concentrations measured during exercise did not suggest that minerals will improve performance during a long term sporting event. As shown in Table VI below, the conflicting results in connection with blood serum concentrations provide no clear suggestion that mineral supplements will positively or negatively affect performance.

TABLE VI
Sample Conflicting Blood Serum Concentrations After Exercise

Blood Serum Concentration After:

| | | Weight | | Bicycle |
|---|---|---|---|---|
| Mineral | Marathon | Training | Triathlon | Ergometrics |
| Ca | No change | | | Increased |
| Mg | Decreased | | | |
| K | | | | |
| Na | | | | |
| Zn | No change | | | |
| Fe | Increased | Decreased | Decreased | |
| Cu | No change | | | Increased |

Having described my invention in such terms as to enable those of skill in the art to understand and use it, and having described the presently preferred embodiments and best mode thereof,

I Claim:

1. A process to improve performance of an individual during a long term sporting event, comprising the steps of
(a) providing in ingestible form a plurality of doses of a first nutritional composition, each dose including a known equivalent concentration of calcium, magnesium, potassium, and zinc;
(b) providing in ingestible form a plurality of doses of a second nutritional composition, each dose of said second nutritional composition including a known equivalent concentration of calcium, magnesium, potassium, sodium, and zinc;
(c) establishing a dietary meal plan including said first nutritional composition for the individual to establish a urine pH in the range of 6.5 to 7.5;
(d) following said dietary meal plan to establish a urine pH in the range of 6.5 to 7.5;
(e) periodically measuring urine pH to confirm a urine pH in the range of 6.5 to 7.5;
(f) during training of the individual prior to the sporting event,
(i) generating data during one or more training intervals indicating changes in urine pH during said training interval,
(ii) administering a selected amount of one or more of said doses of said second nutritional composition after a subsequent training interval, said subsequent training interval extending over a selected period of time,
(iii) generating data indicating changes in urine pH after said second nutritional composition is administered during said subsequent training interval, and
(iv) repeating steps (i) to (iii) to determine a fixed quantity of said doses to maintain urine pH at a selected level in the range of pH 6.5 to pH 7.5 during the sporting event;
(g) beginning to compete in the long term sporting event; and,
(h) at selected intervals during the long term sporting event, ingesting said fixed quantity of said doses to maintain the urine pH at a selected level in the range of pH 6.5 to pH 7.5.
